(19) United States
(12) Patent Application Publication
(10) Pub. No.: US 2024/0423509 A1
(43) Pub. Date: Dec. 26, 2024

(54) NON-INTRUSIVE GLUCOSE MEASUREMENT USING SMART RING WITH PERSONAL CALIBRATION

(71) Applicant: PLUME DESIGN, INC., Palo Alto, CA (US)

(72) Inventors: Miroslav SAMARDZIJA, Mountain View, CA (US); Liem Hieu Dinh VO, San Jose, CA (US)

(21) Appl. No.: 18/338,475

(22) Filed: Jun. 21, 2023

(51) Int. Cl. A61B 5/145; A61B 5/05

(52) U.S. Cl. CPC A61B 5/14532; A61B 5/05; A61B 5/6826; A61B 2560/0223

(57) ABSTRACT

A device may include a body defining an opening including a first module including a receiver/transmitter, the device being configured to perform operations including transmitting a first electromagnetic waves into a digit, receiving a first scattering waves reflected back to the first module, estimating a permittivity based on the first electromagnetic waves and the first scattering waves, and estimating a blood glucose concentration based on the estimated permittivity and a reference dataset and providing the blood glucose concentration as output, the device being configured to be positioned around the digit. The body may also include a second module for receiving and/or transmitting waves, the estimated permittivity being determined based on the waves measured by the second module. The device may also perform a calibration of the device during a first time period to enable the device to estimate the blood glucose concentration of the user during the second time period.

### FIELD

The present disclosure relates to the field of analyte monitoring. More particularly, to an on-body monitoring device for analyte monitoring.

### BACKGROUND

The detection and/or monitoring of glucose levels or other analytes, such as lactate, oxygen, A1C, or the like, is utilized in the treatment of chronic diseases. For example, monitoring of glucose levels in the bloodstream is important to individuals with diabetes. Glucose monitoring includes determining glucose levels in the individual's bloodstream to ensure that levels are being maintained within a safe range, and for determining when insulin may need to be administered. The need for continual monitoring has a direct correlation to glycemic control, but many individuals do not regularly monitor their glucose levels due to a variety of factors including, but not limited to, inconvenience, pain from or fear of needles, costs, or other like factors, which can lead to negative side effects stemming from leaving the underlying chronic illness untreated. Additionally, if these parameters are not regularly monitored, other more serious adverse complications associated with the disease or illness can arise.

### SUMMARY

Traditionally, blood glucose monitoring involves performing invasive fingerstick tests using a needle, up to several times a day. Alternatively, continuous glucose monitoring may use a sensor that requires placing a probe (e.g., needle) under the skin that monitors for glucose concentration, the sensor being in electronically communicable connection with a receiver for displaying glucose levels, storing the measurement data, and/or forwarding the data to a second computing device for analysis. This type of testing poses its own set of risks and can lead to complications, such as an increased risk of infection. Furthermore, individuals may not regularly monitor their blood glucose levels due to the current methodology for blood glucose monitoring.

Various embodiments of the present disclosure may relate to systems, devices, and/or methods for non-invasively monitoring analytes in an individual at an extremity, such as for example, a continuous glucose monitoring system or analyte monitoring system that is worn around a digit of a hand and is capable of monitoring and outputting glucose concentration levels on demand. An analyte monitoring system typically includes a body having an annular shape (e.g., ring-shaped body) and one or more modules for transmitting and/or receiving electromagnetic waves. In some embodiments, the analyte monitoring system may include a data processing unit, such as for example, a processor and a memory. The memory may be a non-transitory computer readable medium having stored thereon one or more instructions executable by the processor to enable the analyte monitoring system to perform one or more operations in accordance with the present disclosure. In some embodiments, the analyte measuring device may include a first module embedded within the annular body for transmitting and receiving the electromagnetic wave signals, the first module in electronically communicable connection with a processing unit configured to control an operation of the first module, to store and process the signals relating to a permittivity of the digital arteries, and to estimate an analyte level or concentration based on the estimated permittivity.

In various embodiments, a device may comprise a ring-shaped body and a first module disposed within the body. The device, e.g., body, may be configured to be worn around a digit of the hand for the purposes of measuring the analyte levels in the individual. The first module may be configured to transmit radio frequency (“RF”) waves into the digit and receive scattering waves reflected back from the digit to enable the device to estimate a permittivity of the digit. For example, the device may estimate a permittivity in the digital arteries. It is to be appreciated by those having ordinary skill in the art that the size and shape of the body of the device is not intended to be limiting and may include any of a plurality of sizes and/or shapes suitable for the device to be positioned around a digit of the wearer, in accordance with the present disclosure. For example, the body of the device may be circumferentially shaped and may include one circumferentially extending side around the annular opening, or the device may be an orthogonal shape having a plurality of sides circumferentially extending around the annular opening.

In some embodiments, the device may be calibrated to account for each individual's specific analyte levels, such as for example, blood glucose levels, to enable the device to estimate permittivity for the specific individual with improved accuracy. To perform the calibration, the device may measure the permittivity of the individual and the device may obtain data corresponding to a glucose concentration level of the individual taken at approximately the same time as the permittivity measurement. The data obtained by the device may be from any of the currently available methods for measuring glucose. For example, the glucose data may be obtained from a fingerstick test measurement. The device performs a correlation determination between the obtained glucose concentration level(s) and the measured permittivity. In some embodiments, the calibration of the device may be performed using a single pair value corresponding to the permittivity measurement of the device and the obtained glucose concentration level. In other embodiments, the calibration may be performed using a plurality of pair values corresponding to the permittivity measurement of the device and the obtained glucose concentration level. In this regard, the correlation may be performed over a period of time, e.g., days, weeks, etc., where the more pair value data available for performing the correlation results in improved accuracy and precision of the permittivity estimation and blood glucose concentration estimation.

The device may be capable of determining the blood glucose levels based on the measured permittivity, in some embodiments. In other embodiments, the device may be in electronically communicable connection with an external computing device, such as for example, a remotely located computing device, and the device may send the measured signals to the external computing device for determining the blood glucose levels based on the measured permittivity of the individual.

The various embodiments of the present disclosure put forward a device and method for non-invasively measuring permittivity in the wearer. The device may be continually worn by the wearer and the device may be configured to measure the permittivity throughout the day as determined by the wearer without having to perform invasive fingerstick tests. Accordingly, the number of fingerstick tests that may need to be performed for monitoring purposes is reduced, which may aid those with fears of needles or those that wish to avoid discomfort from having to perform frequent fingerstick tests. Because of the continual nature of blood glucose monitoring needs, and the young age of many patients, the ability to wear a non-invasive monitoring device that measures permittivity can significantly improve the day-to-day experience of those suffering from chronic illnesses.

In some embodiments, a device includes a body defining an annular-shaped opening, the body including a first module including a receiver/transmitter, and the device performs one or more operations including transmit, by the first module, a first electromagnetic waves into a digit of a user, receive, by the first module, a first scattering waves reflected back to the first module, estimate a permittivity based on the first electromagnetic waves and the first scattering waves, and estimate, during a second time period, a blood glucose concentration based on the estimated permittivity and a reference dataset and providing the blood glucose concentration as output, the device being configured to be worn by the user such that the body is positioned around the digit.

In some embodiments, the body further includes a second module including a receiver, the second module being opposite the body from the first module.

In some embodiments, the device further performs operations including receive, by the second module, a second scattering waves passing through the digit from the first module, and estimating the permittivity is further based on the second scattering waves received by the second module.

In some embodiments, the second module further includes a transmitter, and the device further performs operations including transmit, by the second module, a second electromagnetic waves into a digit of a user, and receive, by the second module, a third scattering waves reflected back to the second module, and estimating the permittivity is further based on the second electromagnetic waves and the third scattering waves.

In some embodiments, estimating the permittivity further includes determining a complex permittivity corresponding to an amplitude and phase characteristics of the scattering waves.

In some embodiments, the device further performs operations including perform a calibration of the device during a first time period to enable the device to estimate the blood glucose concentration of the user during the second time period.

In some embodiments, performing the calibration further includes estimate the permittivity of the user during one or more intervals of the first time period, obtain one or more reference values corresponding to glucose concentration values measured during the one or more intervals of the first time period, and produce a model including a reference electromagnetic wave plot and a scattered wave plot, the estimated blood glucose concentration being determined during the second time period after the first time period and based on the model.

In some embodiments, the first time period includes a plurality of permittivity measurements and corresponding reference values obtained during a plurality of measurement intervals.

In some embodiments, the estimated permittivity of the user is further based on a vacuum permittivity.

In some embodiments, a frequency range of the electromagnetic waves includes an RF frequency range.

In some embodiments, a system includes a body defining an annular-shaped opening, the body is configured to be worn on a hand of a user such that a digit of the hand extends through the opening, a first module including a receiver/transmitter configured to transmit electromagnetic waves within a certain frequency range into the digit and receive scattering waves reflected back to the first module, a computing device including a processor, and a non-transitory computer readable medium having stored thereon one or more instructions executable by the processor to enable the first module to perform one or more operations including transmit, by the first module, a first electromagnetic waves into a digit of a user, receive, by the first module, a first scattering waves reflected back to the first module, estimate a permittivity based on the first electromagnetic waves and the first scattering waves, and estimate, during a second time period, a blood glucose concentration based on the estimated permittivity and a reference dataset and providing the blood glucose concentration as output.

In some embodiments, the body further includes a second module including a receiver, the second module is located in the body opposite the first module, the computing device causes the second module to perform operations including receive, by the second module, a second scattering waves passing through the digit from the first module, estimating the permittivity is further based on the second scattering waves received by the second module.

In some embodiments, the second module further includes a transmitter, the device further performs operations including transmit, by the second module, a second electromagnetic waves into a digit of a user, and receive, by the second module, a third scattering waves reflected back to the second module, and estimating the permittivity is further based on the second electromagnetic waves and the third scattering waves.

In some embodiments, the computing device further performs operations including perform a calibration of the device during a first time period to enable the device to estimate the blood glucose concentration of the user during a second time period, the calibration including estimate the permittivity of the user during one or more intervals of the first time period, obtain one or more reference values corresponding to glucose concentration values measured during the one or more intervals of the first time period, and producing a model including a reference electromagnetic wave plot and a scattered wave plot, the estimated blood glucose concentration being determined during the second time period after the first time period and based on the model.

In some embodiments, a frequency range of the electromagnetic waves includes an RF frequency range.

In some embodiments, a method for measuring a glucose concentration level using a measurement device configured to be worn on a hand of a user, the device including a first module including a receiver/transmitter, the method including transmitting, by the first module, a first electromagnetic waves into a digit of a user, receiving, by the first module, a first scattering waves reflected back to the first module, estimating a permittivity based on the first electromagnetic waves and the first scattering waves, and estimating, during a second time period, a blood glucose concentration based on the estimated permittivity and a reference dataset and providing the blood glucose concentration as output.

In some embodiments, the device further includes a second module including a receiver, the method further including receiving, by the second module, a second scattering waves passing through the digit from the first module, estimating the permittivity is further based on the second scattering waves received by the second module.

In some embodiments, the second module further includes a transmitter, the method further including transmitting, by the second module, a second electromagnetic waves into a digit of a user, and receiving, by the second module, a third scattering waves reflected back to the second module, estimating the permittivity is further based on the second electromagnetic waves and the third scattering waves.

In some embodiments, the method further includes performing a calibration of the device during a first time period to enable the device to estimate the blood glucose concentration of the user during the second time period.

In some embodiments, performing the calibration further includes estimating the permittivity of the user during one or more intervals for the first time period, obtaining the reference dataset including one or more reference values corresponding to glucose concentration values measured during the one or more intervals for the first time period, and producing a model including a reference electromagnetic wave plot and a scattered wave plot based on the reference dataset and the permittivity estimated during the first time period, the estimated blood glucose concentration being determined during the second time period after the first time period and based on the model.

### BRIEF DESCRIPTION OF THE DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIG. 1 is a perspective view of a non-limiting example embodiment of a device, according to some embodiments.

FIG. 2 is a side view of the device, according to some embodiments.

FIG. 3 is a second side view of the device, according to some embodiments.

FIG. 4 is a block diagram of analyte monitoring performed by the device, according to some embodiments.

FIGS. 5a-5c are graphical diagrams for measuring permittivity at the device, according to some embodiments.

FIG. 6 is an example system, according to some embodiments.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

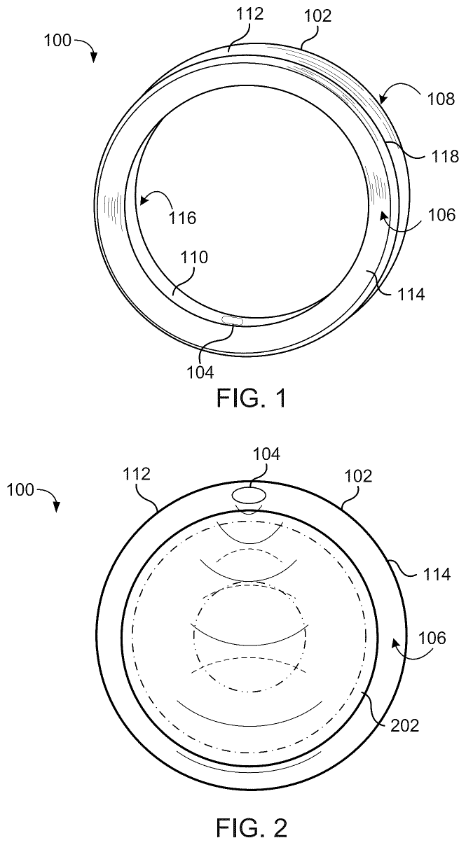

FIG. 1 illustrates an perspective view of a non-limiting example of a device 100, according to some embodiments. FIG. 2 illustrates an exposed side view of the device 100 of FIG. 1, according to some embodiments. Unless specifically referenced, FIGS. 1 and 2 will be described collectively.

A device 100 includes a body 102 and a module 104. The body 102 includes a first side 106 and a second side 108 opposite the first side 106, and the body 102 may define an annular-shaped opening extending therethrough from the first side 106 to the second side 108. Accordingly, the body 102 of device 100 may be configured to be worn around a digit 202 (e.g., finger) of a hand of an individual. As such, the body 102 may be ring-shaped, in some embodiments. In other embodiments, the body 102 may include one or more other shapes. It is to be appreciated by those having ordinary skill in the art that the shape and size of the body 102 of the device 100 is not intended to be limiting and the device 100 may include any of a plurality of shapes and sizes so long as the device 100 can be capable of being placed around a digit 202 of an individual for measuring permittivity, in accordance with the present disclosure.

The body 102 includes a surface 110 defining an inner circumference, the surface 110 configured to contact a skin of the digit 202. The body 102 also includes surface 112 defining an outer circumference. In some embodiments, the surface 112 may be substantially rounded and extend from the first side 106 to the second side 108. The body 102 may further include a sidewall 114 and a sidewall 116 opposite sidewall 114 extending between the surface 110 and the surface 112. The sidewall 114 being adjacent the module 104 and the sidewall 116 being adjacent the second side 108. Additionally, the body 102 may further include one or more bevels 118. For example, in some embodiments, and as shown in FIG. 1, the body 102 may include a bevel 118 located between sidewall 114 and the surface 112. In some embodiments, the body 102 may also include a bevel 118 located between sidewall 116 and surface 112. In other embodiments, the body 102 may also include bevels 118 located between the sidewall 114 and the surface 110 or between the sidewall 116 and the surface 110.

Device 100 also include module 104. The module 104 may be embedded in the body 102. In some embodiments, the module 104 may be located adjacent the surface 110 for transmitting/receiving electromagnetic waves, as will be further described herein. In other embodiments, the module 104 may be located at the surface of surface 110 and configured to contact the skin of the digit 202. The module 104 includes a transmitter/receiver device for transmitting electromagnetic waves into the digit 202 of the hand. In some embodiments, the electromagnetic waves may be one or more types of electromagnetic waves. In other embodiments, the electromagnetic waves may include radio frequency (“RF”) waves. In some embodiments, the electromagnetic waves may be RF waves. The module 104 may also be capable of measuring the scattering waves reflected back to the module 104 from the digit 202 of the hand. In this regard, the device 100 utilizes the transmit and measured scattering waves to estimate the permittivity of the body of the individual wearing the device 100. In this regard, the device 100 estimates both the amplitude and phase of the complex permittivity, which is then correlated to glucose concentration in the bloodstream, as will be further described herein.

The device 100 and/or the module 104 may further includes a processor 120 and a memory 122. The processor 120 may control an operation of the device 100. For example, the processor 120 may control the transmitter/receiver device for transmitting electromagnetic waves into the digit 202. The memory 122 may be a non-transitory computer readable media having stored thereon one or more instructions executable by the processor to enable the device 100 to perform one or more operations for estimating a glucose concentration in the body of the wearer, in accordance with the present disclosure.

The device 100 may also include one or more other components that enable the device 100 to estimate the complex permittivity and to correlate the glucose concentration in the wearer based on the permittivity, in accordance with the present disclosure. In some embodiments, the device 100 may include one or more other components for enabling the device 100 to be placed in electronically communicable connection with one or more other computing devices to send and receive information between the device 100 and the external computing device. For example, the module 104 may include a input/output component for connecting to an external computing device. In another example, the device 100 may obtain a reference dataset from the external computing device for determining a correlation between the estimated permittivity and the blood glucose concentration. In yet another example, the device 100 may send the measured scattering waves, the estimated permittivity, or other data to the external computing device for analysis, displaying, etc.

Referring to FIG. 2, the module 104 may include a transmitter/receiver device for transmitting electromagnetic waves, such as for example, RF waves, into the digit 202 of the hand. The module 104 may also be capable of measuring the scattering waves that reflect back and pass through the digit 202 of the wearer. In some embodiments, the module 104 may include a vector network analyzer (“VNA”) for measuring electrical parameters, such as for example, RF waves and the scattering waves reflected back from the digit 202 of the hand. In this regard, the module 104 may include one or more filters, antennas, power amplifiers, or the like, that enable the module 104 and the device 100 to transmit and receive electromagnetic wave signals in accordance with the present disclosure. The module 104 detects changes to the output signal, e.g., scattering waves, and compares that to the source signal, e.g., emitted RF waves, and measures that amplitude and phase responses caused by the module 104 and caused by the waves passing through or being reflected back from the digit 202 of the wearer.

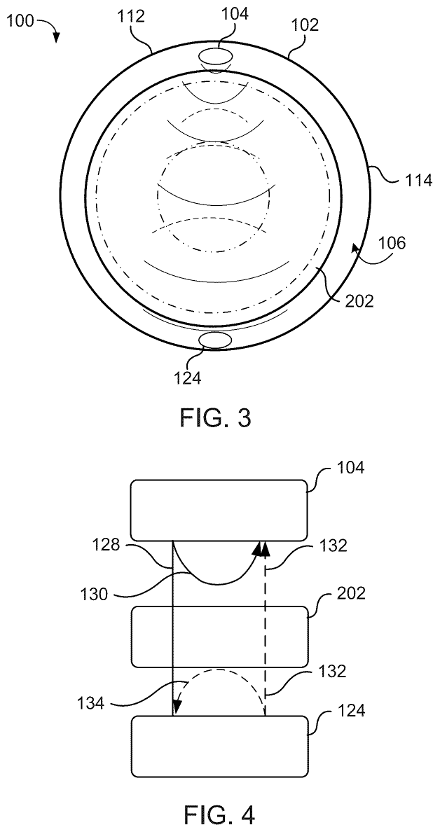

FIG. 3 illustrates a second side view of the device 100, according to some embodiments.

The device 100 may include module 124. Module 124 may be located in the body 102 of device 100 opposite from module 104. In some embodiments, the module 124 may be embedded in the body 102. In some embodiments, the module 124 may be located adjacent the surface of surface 110. In other embodiments, the module 124 may be located at the surface of surface 110. The module 124 may include a receiver for receiving the electromagnetic waves transmitted by the module 104 to enable the device 100 to estimate the permittivity by measuring the electromagnetic waves that pass through the digit 202 and the scattering waves that reflect back to module 104. In some embodiments, the module 124 may also be a transmitter capable of transmitting electromagnetic waves into and through the digit 202 of the wearer, similar to module 104. In this regard, the module 124 may also be capable of transmitting electromagnetic waves and measuring the scattering waves that reflect back from the digit 202 of the wearer. Accordingly, in some embodiments, the module 104, the module 124, or both may transmit and receive electromagnetic waves for the purposes of estimating the permittivity and for estimating the blood glucose concentration of the wearer.

FIG. 4 illustrates a block diagram of analyte monitoring performed by the device 100, according to some embodiments.

In some embodiments, to estimate the permittivity in the digit 202 (e.g., finger) of the wearer, the module 104 transmits RF waves into the digit 202. The RF waves pass through the digit 202 such as they would pass through a dielectric having a certain permittivity. In this regard, the permittivity as estimated by device 100 may be a function of relative permittivity and vacuum permittivity. The relative permittivity of the digit 202 is estimated by measuring one or more scattering parameters resulting from transmitting the electromagnetic waves into the digit 202 and the waves being reflected back or passing through the digit 202. As used herein, the term “vacuum permittivity” refers to a physical constant corresponding to the value of absolute dielectric permittivity of a classical vacuum.

The one or more scattering parameters may correspond to transmitted coefficients measured by the module 104, the module 124, or both during the transmitter and receiver phase of measuring the permittivity for the purposes of estimating the blood glucose concentration. The transmitted coefficients may include a coefficient 128 corresponding to the RF waves that are transmitted by module 104 and which pass through the digit 202 and are measured by the module 124. The transmitted coefficients may include a coefficient 130 corresponding to the scattering waves that are reflected back from the digit 202 from the RF waves transmitted by module 104 and which are measured by the receiver at module 104. In some embodiments, the transmitted coefficients may also include a coefficient 132 corresponding to RF waves that are transmitted by module 124 and which pass through the digit 202 and are measured by the receiver of module 104. The transmitted coefficients may also include a coefficient 134 corresponding to the scattering waves reflected back from digit 202 from the RF waves transmitted by module 124 and which are measured by the receiver at module 124.

In some embodiments, the device 100 may only include module 104 and the device 100 may estimate the permittivity based on coefficient 130 received at module 104. In some embodiments, the device 100 may include module 104 and module 124 may only include a receiver therein, and the device 100 may estimate the permittivity based on coefficient 128 and coefficient 130. In other embodiments, the device 100 may include module 104 and module 124, both having a transmitter/receiver located therein, and the device 100 may estimate the permittivity based on coefficient 128, coefficient 130, coefficient 132, and coefficient 134 received by the module 104 and module 124, respectively.

The device 100 and/or the module 104 measures the electromagnetic waves that either pass through the digit 202 or reflect back from the digit 202 and estimates the permittivity of the digit 202. The permittivity of the digit 202 may be a function of glucose, finger size, body type, etc., in some embodiments. In this regard, the more scattering parameters that are considered, e.g., through the transmitted coefficients, the more accurately the permittivity of the digit 202 can be estimated. Additionally, permittivity is more accurately estimated when an amplitude and phase is taken account for the measured electromagnetic signals. For example, the amplitude of coefficient 130 and the phase angle of coefficient 130 may be determined by the module 104 based on receiving the scattering waves at the receiver of module 104.

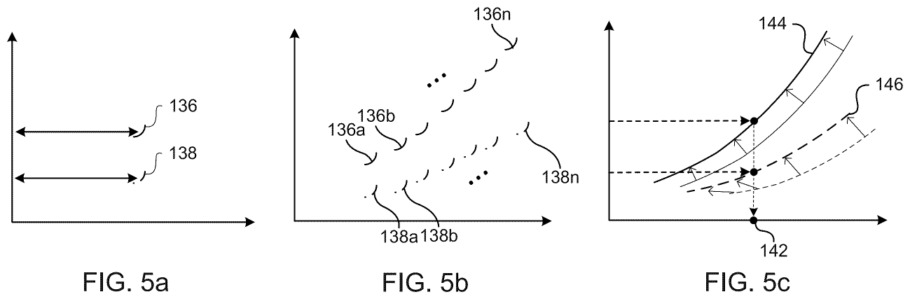

FIG. 5a illustrates a graphical diagram for measuring permittivity at the device 100, according to some embodiments. FIG. 5b illustrates a second graphical diagram for measuring permittivity at the device 100, according to some embodiments. Unless specifically referenced, FIGS. 5a and 5b will be described collectively.

The device 100 may be capable of estimating a complex permittivity based on characteristics of the waves received at the respective modules. In this regard, the scattering waves received at module 104, module 124, or both, may have well defined amplitude(s) and phase(s). As used herein, “complex permittivity,” refers to effects on the electromagnetic wave's amplitude and phase during transmit, pass-through, and/or reflected stages. Accordingly, the device 100 measures the complex permittivity of the digit 202 by transmitting the RF waves into the digit 202 and measuring the amplitude and phase of the scattering waves.

Referring to FIG. 5a, for every scattering wave, there may be two data points 136 and 138 measured by the respective module 104, module 124, or both. The data point 136 may be the permittivity measured by the device 100 corresponding to the waves that pass through the digit 202. In some embodiments, the data point 136 may correspond to the RF waves output by the module 104. In other embodiments, the data point 136 may correspond to the RF waves output by module 104 and received at module 124. In other embodiments, the data point 136 may correspond to the RF waves output by module 124 and received by module 104. In yet other embodiments, the data point 136 may correspond to the RF waves output by module 124.

The data point 138 may be the waves reflected back from the digit 202 and received by the respective one of module 104 and/or module 124. In some embodiments, the data point 138 may correspond to the scattering waves received at module 104. In other embodiments, the data point 138 may correspond to scattering waves received by module 124.

Referring to FIG. 5b, as the wearer engages in certain activities, such as consuming certain food or liquids that trigger an insulin response, the wearer's blood glucose concentration may change. As a result, the permittivity measured by device 100 may also change. The device 100 may therefore continually, intermittently, or any range or subrange therebetween, measure the permittivity of the wearer and output results based on the measurement. The complex permittivity measured for the wearer may be represented as data points 136 a-136 n and data points 138 a-136 n, hereinafter referred to as data points 136 and data points 138 respectively. As shown in FIG. 5b, the data points 136 and data points 138 increase during the period of measurement shown in the graph. As such, the device 100 may output data corresponding to the estimated blood glucose concentration levels to the user, such as for example, to the external computing device of the user. For example, the device 100 may be in electronically communicable connection with a mobile phone of the user, and the device 100 may transmit the measured blood glucose concentrations, the measured permittivity, other data corresponding to the permittivity or blood glucose (e.g., metadata), or any combinations thereof, to the mobile device and the mobile device may alert the user when the blood glucose levels exceed one or more limits or is approaching one or more limits.

As permittivity, and corresponding blood glucose concentrations, of the wearer increases or decreases throughout the day based on the user's activities, measuring permittivity one or more times throughout the day may be beneficial for effective treatment. As such, the device 100 may measure the permittivity once per day or at regular intervals based on a setting determined by the user. In some embodiments, the device 100 may measure permittivity only at intervals determined by the user. In other embodiments, the device 100 may measure permittivity at one or more intervals as established by the user. In yet other embodiments, the device 100 may measure permittivity at the one or more intervals as established by the user.

The device 100 may also measure permittivity at other times outside the time intervals established by the user. In this regard, the device 100 may also be capable of determining the blood glucose concentration based on measurements taken outside one of the intervals established by the user to determine whether the blood glucose concentration exceeds one or more limits established by the user. The device 100 may either store these other results in the memory for a period of time. The device 100 may also transmit these results to the external computing device for reporting, data tracking, or other like purposes. For example, if the glucose measurement exceeds a threshold established by the device 100 and/or the user, the device 100 may send the glucose results to the external computing device to alert the user. In another example, if the glucose measurement does not exceed a threshold, the device 100 may either store the measured values in the memory [ref] or may discard the data associated therewith.

FIG. 5c illustrates a graphical diagram of a model for calibrating the device 100, according to some embodiments.

Each individual may have unique biological and physiological characteristics which affect the blood glucose measurement estimation (e.g., finger size, body type, weight, blood pressure, sodium levels, etc.) performed by the device 100. As such, the device 100 may perform a calibration during a first time period to account for the unique characteristics of each individual wearer such as to enable the device 100 to accurately estimate the permittivity and corresponding blood glucose concentration of each wearer during a second time period. The device 100 may therefore obtain a reference dataset corresponding to reference blood glucose measurements values obtained using one of the other available testing methods and the reference dataset may be leveraged by the device 100 to determine a correlation between a measured permittivity and an estimated blood glucose concentration based on a reference dataset during the second time period.

The reference dataset may include one or more reference values 142 corresponding to blood glucose concentration values measured using any of the other known measuring methods during the first time period. In this regard, the reference values 142 and the permittivity of the wearer as measured by device 100 are obtained during the first time period. In some embodiments, the reference values 142 may be obtained within a certain time period as the permittivity measured by the device 100. In other embodiments, the reference values may be obtained at substantially the same time as when the device 100 measures permittivity.

These one or more reference values 142 may be obtained by device 100 by one or more approaches. For example, the user, e.g., the wearer of device 100, may perform fingerstick testing within the time period of the permittivity measurement performed by the device 100 and the user may input the blood glucose level into the device 100 or another computing device in electronically communicable connection with device 100. In some embodiments, the device 100 may be calibrated with one reference value. In other embodiments, the device 100 may be calibrated with one or more reference values. It is to be appreciated by those having ordinary skill in the art that the type of method for measuring glucose which may be utilized for correlating the estimated permittivity by the device 100 is not intended to be limiting and may include the foregoing methods or other available methods for measuring blood glucose concentration.

During a calibration phase, for each permittivity value, e.g., data points 136 and data points 138, measured by device 100, the blood glucose concentration of the wearer may also be measured using any of the other known methods and obtained by the device 100 as reference values 142, which may be provided to the device 100. For example, the external computing device may receive the one or more reference values 142 as input, which may then be transmitted to the device 100. In some embodiments, the user may input the reference value(s) into the device 100. In other embodiments, the external computing device may obtain the reference values, such as for example, based on inputs from the user, which may transmit the reference data to the device 100. For example, the user may enter the reference values into a user interface presented on a display of the external computing device, which the user may then upload to the device 100. In some embodiments, the reference values may be obtained from other methods including, but not limited to, fingerstick tests, continual glucose monitoring, and other like methods.

In this regard, calibration of device 100 using one of the other methods for measuring glucose may include pricking an extremity of the wearer/user to draw blood from the finger or to insert a sensor into the extremity. However, this other testing may be performed so as to enable the device 100 to obtain the measured glucose values corresponding to reference values for calibrating the device 100. After the reference dataset is obtained by the device 100, further invasive testing may be avoided and the device 100 may be capable of measuring the permittivity of the digit 202 and estimated the corresponding glucose measurement associated therewith without invasive testing methods.

To enable the device 100 to estimate the blood glucose concentration correlating to permittivity measured at a second time period after the first time period, the device 100 may obtain the one or more reference values 142 and may generate one or more models for determining the correlation between measured permittivity and estimated blood glucose concentration. Referring to FIG. 5c, the model may include line 144 and line 146. Line 144 and line 146 may correspond to a modeled plotting of RF waves and scattering waves, respectively, based on the calibration (e.g., translation). In this regard, the line 144 and line 146 may be modeled by device 100 based on performing a calibration on data points 136 and 138, such that permittivity values measured by device 100 during the second time period that fall along the respective line 144 and line 146 correspond to modeled glucose measurement values. As such, at the second time period, the device 100 may measure the permittivity of the wearer and compare the measured permittivity to the one or more models, such as a model including line 144 and line 146 therein, to determine the corresponding blood glucose concentration based on the measured permittivity. The device 100 may then provide the blood glucose concentration value as output to the user and/or the external computing device.

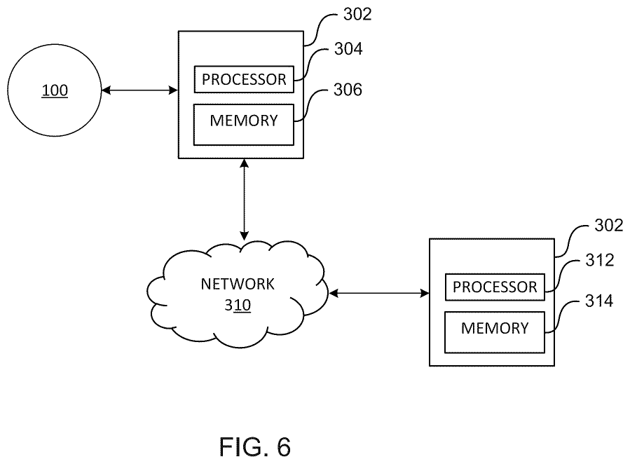

FIG. 6 illustrates an example system 300, according to some embodiments.

The system 300 may include device 100 and computing device 302, the device 100 being in electronically communicable connection with the computing device 302. For example, the device 100 may include one or more components for communicating with computing device 302 via Bluetooth or other like communication protocols. However, it is to be appreciated by those having ordinary skill in the art that the types of protocols that may be utilized for the device 100 to communicate with the computing device 302 is not intended to be limiting and may include any of a plurality of protocols in accordance with the present disclosure.

The computing device 302 may also include a processor 304 and a memory 306. The processor 304 may execute one or more instructions stored in the memory 306. In this regard, the processor 304 may enable the computing device 203 to communicate with device 100, send and receive data between device 100 and computing device 302, and may also estimate the blood glucose concentration based on permittivity levels obtained by from device 100 and/or based on the glucose measurements taken during the first time period, in accordance with the present disclosure. For example, in some embodiments, the computing device 302 may obtain the reference values 142 and the computing device 302 may store the reference values 142 in the memory 306 until the calibration of device 100 is performed. In other embodiments, the computing device 302 may include instructions stored in the memory 306 and executable by the processor 304 for the computing device 302 to perform the calibration of device 100 as described in the foregoing and for the computing device 302 to generate the one or more models for estimating the blood glucose levels based on measured permittivity.

As such, in some embodiments, the computing device 302 may determine the one or more models and send the model data to the device 100 to enable the device 100 to measure the blood glucose concentrations of the wearer. In other embodiments, the computing device 302 may obtain data from device 100 corresponding to the measured permittivity of the wearer and compare the permittivity to the one or more models and determine the blood glucose concentration levels of the wearer. Accordingly, the computing device 302 may display the results on a display associated with the computing device 302 and/or may transmit, or forward, the measurement results to another computing device 308.

Additionally, the computing device 302 may also be placed in electronically communicable connection with one or more other computing devices 308. Additionally, in some embodiments, the computing device 302 may be in electronically communicable connection with the device 100 and/or the one or more computing device 308 through a network 310. In some embodiments, the computing device 308 may be associated with a medical professional, such as a doctor of the wearer of the device 100, and the computing device 302 may transmit the measured values to the computing device 308 and may also obtain one or more user settings from the computing device 308. In other embodiments, the computing device 308 may be one or more processing machines including one or more processors 312 and one or more memories 314 for obtaining data and processing said data in accordance with instructions stored therein. For example, the computing device 308 may obtain the data corresponding to the permittivity values as measured by device 100 and the blood glucose levels, e.g., reference values 142, and produce one or more models based on the inputs. The computing device 308 may then transmit the one or more models to the computing device 302, such as to enable the computing device 302 and/or device 100 to measure the blood glucose concentrations of the user wearing the device 100 during the second time period.

All prior patents and publications referenced herein are incorporated by reference in their entireties.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a.” “an,” and “the” include plural references. The meaning of “in” includes “in” and “on.”

As used herein, the term “between” does not necessarily require being disposed directly next to other elements. Generally, this term means a configuration where something is sandwiched by two or more other things. At the same time, the term “between” can describe something that is directly next to two opposing things. Accordingly, in any one or more of the embodiments disclosed herein, a particular structural component being disposed between two other structural elements can be:
disposed directly between both of the two other structural elements such that the particular structural component is in direct contact with both of the two other structural elements;
disposed directly next to only one of the two other structural elements such that the particular structural component is in direct contact with only one of the two other structural elements;
disposed indirectly next to only one of the two other structural elements such that the particular structural component is not in direct contact with only one of the two other structural elements, and there is another element which juxtaposes the particular structural component and the one of the two other structural elements; disposed indirectly between both of the two other structural elements such that the particular structural component is not in direct contact with both of the two other structural elements, and other features can be disposed therebetween; or any combination(s) thereof.

As used herein “embedded” means that a first material is distributed throughout a second material.

What is claimed is:

1. A device comprising:
a body defining an annular-shaped opening, the body comprising:
a first module comprising:
a receiver/transmitter; and
wherein the device performs one or more operations comprising:
transmit, by the first module, a first electromagnetic waves into a digit of a user,
receive, by the first module, a first scattering waves reflected back to the first module,
estimate a permittivity based on the first electromagnetic waves and the first scattering waves, and
estimate, during a second time period, a blood glucose concentration based on the estimated permittivity and a reference dataset and providing the blood glucose concentration as output;
wherein the device is configured to be worn by the user such that the body is positioned around the digit.

2. The device of claim 1, wherein the body further comprises:
a second module comprising:
a receiver,
wherein the second module is opposite the body from the first module.

3. The device of claim 2, wherein the device further performs operations comprising:
receive, by the second module, a second scattering waves passing through the digit from the first module,
wherein estimating the permittivity is further based on the second scattering waves received by the second module.

4. The device of claim 2, wherein the second module further comprises:
a transmitter, and
wherein the device further performs operations comprising:
transmit, by the second module, a second electromagnetic waves into a digit of a user, and
receive, by the second module, a third scattering waves reflected back to the second module;
wherein estimating the permittivity is further based on the second electromagnetic waves and the third scattering waves.

5. The device of claim 1, wherein estimating the permittivity further comprises:
determining a complex permittivity corresponding to an amplitude and phase characteristics of the scattering waves.

6. The device of claim 1, wherein the device further performs operations comprising:
perform a calibration of the device during a first time period to enable the device to estimate the blood glucose concentration of the user during the second time period.

7. The device of claim 6, wherein performing the calibration further comprises:
estimate the permittivity of the user during one or more intervals of the first time period,
obtain one or more reference values corresponding to glucose concentration values measured during the one or more intervals of the first time period, and
produce a model comprising a reference electromagnetic wave plot and a scattered wave plot,
wherein the estimated blood glucose concentration is determined during the second time period after the first time period and based on the model.

8. The device of claim 6, wherein the first time period comprises a plurality of permittivity measurements and corresponding reference values obtained during a plurality of measurement intervals.

9. The device of claim 1, wherein the estimated permittivity of the user is further based on a vacuum permittivity.

10. The device of claim 1, wherein a frequency range of the electromagnetic waves comprises an RF frequency range.

11. A system comprising:
a body defining an annular-shaped opening,
wherein the body is configured to be worn on a hand of a user such that a digit of the hand extends through the opening;
a first module comprising:
a receiver/transmitter configured to transmit electromagnetic waves within a certain frequency range into the digit and receive scattering waves reflected back to the first module; and
a computing device comprising:
a processor, and
a non-transitory computer readable medium having stored thereon one or more instructions executable by the processor to enable the first module to perform one or more operations comprising:
transmit, by the first module, a first electromagnetic waves into a digit of a user,
receive, by the first module, a first scattering waves reflected back to the first module,
estimate a permittivity based on the first electromagnetic waves and the first scattering waves, and
estimate, during a second time period, a blood glucose concentration based on the estimated permittivity and a reference dataset and providing the blood glucose concentration as output.

12. The system of claim 11, wherein the body further comprises:
a second module comprising:
a receiver,
wherein the second module is located in the body opposite the first module; and
wherein the computing device causes the second module to perform operations comprising:
receive, by the second module, a second scattering waves passing through the digit from the first module;
wherein estimating the permittivity is further based on the second scattering waves received by the second module.

13. The system of claim 12, wherein the second module further comprises:
a transmitter, and
wherein the device further performs operations comprising:
transmit, by the second module, a second electromagnetic waves into a digit of a user, and
receive, by the second module, a third scattering waves reflected back to the second module;
wherein estimating the permittivity is further based on the second electromagnetic waves and the third scattering waves.

14. The system of claim 11, wherein the computing device further performs operations comprising:
perform a calibration of the device during a first time period to enable the device to estimate the blood glucose concentration of the user during a second time period, the calibration comprising:
estimate the permittivity of the user during one or more intervals of the first time period,
obtain one or more reference values corresponding to glucose concentration values measured during the one or more intervals of the first time period, and producing a model comprising a reference electromagnetic wave plot and a scattered wave plot;
wherein the estimated blood glucose concentration is determined during the second time period after the first time period and based on the model.

15. The system of claim 11, wherein a frequency range of the electromagnetic waves comprises an RF frequency range.

16. A method for measuring a glucose concentration level using a measurement device configured to be worn on a hand of a user, the device comprising a first module including a receiver/transmitter, the method comprising:
transmitting, by the first module, a first electromagnetic waves into a digit of a user;
receiving, by the first module, a first scattering waves reflected back to the first module;
estimating a permittivity based on the first electromagnetic waves and the first scattering waves; and
estimating, during a second time period, a blood glucose concentration based on the estimated permittivity and a reference dataset and providing the blood glucose concentration as output.

17. The method of claim 16, wherein the device further comprises a second module including a receiver, the method further comprising:
receiving, by the second module, a second scattering waves passing through the digit from the first module;
wherein estimating the permittivity is further based on the second scattering waves received by the second module.

18. The method of claim 17, wherein the second module further comprises a transmitter, the method further comprising:
transmitting, by the second module, a second electromagnetic waves into a digit of a user; and
receiving, by the second module, a third scattering waves reflected back to the second module;
wherein estimating the permittivity is further based on the second electromagnetic waves and the third scattering waves.

19. The method of claim 18, further comprising:
performing a calibration of the device during a first time period to enable the device to estimate the blood glucose concentration of the user during the second time period.

20. The method of claim 19, wherein performing the calibration further comprises:
estimating the permittivity of the user during one or more intervals for the first time period;
obtaining the reference dataset comprising one or more reference values corresponding to glucose concentration values measured during the one or more intervals for the first time period; and
producing a model comprising a reference electromagnetic wave plot and a scattered wave plot based on the reference dataset and the permittivity estimated during the first time period;
wherein the estimated blood glucose concentration is determined during the second time period after the first time period and based on the model.
